(19) United States
(12) Patent Application Publication
(10) Pub. No.: US 2016/0289142 A1
(43) Pub. Date: Oct. 6, 2016

(54) Natural Gas Decarbonization Process for Production of Zero-Emission Benzene and Hydrogen from Natural Gas

(71) Applicant: Pioneer Energy Inc., Lakewood, CO (US)

(72) Inventors: Robert Zubrin, Golden, CO (US); Mark Berggren, Golden, CO (US)

(73) Assignee: Pioneer Energy, Lakewood, CO (US)

(21) Appl. No.: 15/081,042

(22) Filed: Mar. 25, 2016

Related U.S. Application Data
(60) Provisional application No. 62/140,162, filed on Mar. 30, 2015.

(51) Int. Cl. C07C 2/76; C10G 1/04; C07C 1/12

(52) U.S. Cl. CPC C07C 2/76; C07C 1/12; C10G 1/047; C07C 2529/48

(57) ABSTRACT

A process for producing aromatic hydrocarbons from methane or natural gas is described. The process operates by contacting the methane or natural gas along with hydrogen recycled in the system over a catalyst at elevated temperatures. During each pass over the catalyst, methane or natural gas is converted to benzene, toluene, naphthalene, and other aromatic compounds. The process can be used to produce zero-emission hydrogen, which can be used for generation of zero-emission electricity, generation of steam for use in extraction of heavy oil and oil sands, or for other purposes. In addition, benzene, an aromatic hydrocarbon, is produced, which is a readily-transportable and valuable chemical commodity and a fuel component, which can be used to displace petroleum-based gasoline and diesel, leading to additional above-ground GHG emission reductions. As a by-product, hydrogen is produced, which is used to produce zero-emission, high-quality steam and/or carbon-free electricity for above-ground facilities.

### RELATED APPLICATIONS

This application claims the benefit of U.S. Provisional Application No. 62/140,162 filed on 30 Mar. 2015 which is incorporated herein by reference.

### BRIEF DESCRIPTION OF DRAWINGS

FIG. 1 shows a Natural Gas Decarbonization Process Block Flow Diagram

FIG. 2 shows a schematic representation of a configuration of the natural gas to aromatics Methane Aromatization System according to one embodiment of the invention.

FIG. 3 shows the effect of inlet hydrogen concentration on benzene yield for 6% Mo-0.7% In/HZSM-5

FIG. 4 shows a comparison of the benzene yields at 700° C. for methane-activated Small Batch, hydrogen-regenerated Small Batch, methane-activated Large Batch, and butane-activated Large Batch 6% Mo-0.7% In/HZSM-5

### DETAILED DESCRIPTION OF THE INVENTION

An important aspect of the present invention is a process to produce low or zero-emission steam, clean electricity, and benzene from natural gas to decrease or virtually eliminate carbon dioxide emissions associated with steam generation for oil production from tar sands or other purposes as needed.

In one aspect, the Natural Gas Decarbonization Process utilizes an aromatization reaction to generate a valuable benzene product and high-quality steam.

The current method for steam generation requires combustion of natural gas in an amount equivalent to about 20 percent of the energy content contained in the 33,000 barrels per day of extracted oil—the existing method results in daily CO₂ emissions of about 2100 metric tons. The following reaction (1) is employed:

6CH₄→C₆H_{6(g)}±9H₂ ΔH=603 kJ@900° C. (1)

In other embodiments, the present invention is used in space travel and colonization. In one embodiment, it is employed to address the need for low hydrogen content fuel for return flights from Mars, whether for sample return or human return, a process was developed to convert the carbon dioxide in the Martian atmosphere into aromatic hydrocarbons. This process combined the carbon dioxide from a simulated Martian atmosphere with hydrogen, which would be brought from Earth on a real mission, over a Sabatier reaction catalyst to make methane and water. By use of a recycling system, the methane was then completely converted into aromatic compounds, primarily benzene, over a molybdenum impregnated aluminosilicate molecular sieve catalyst at 600-800° C. Byproduct hydrogen was recycled after membrane separation to make more methane. This entire process was named the Methane to Aromatics on Mars or METAMARS process and finds application to methane conversion on Earth.

In other embodiments the present invention is used to tap remote sources of natural gas and hydrocarbons. More than 2,500 trillion cubic feet of proved natural gas reserves are stranded too far from market for economic recovery (Chevron). Storage and transportation are key elements in the economic recovery of natural gas. Conversion of natural gas in remote locations to an economically transportable liquid product will enable the ultimate utilization of these huge resources. Technologies to produce valuable chemicals from natural gas are also of great interest to major petrochemical companies worldwide. The present benzene production process very effectively addresses converting natural gas to an easily transportable liquid while also generating a valuable chemical product.

In one embodiment, the conversion of methane and higher alkanes to benzene is carried out over a catalyst, such as molybdenum-ZSM, at temperatures above 700° C. at near atmospheric pressure. The reaction produces nine moles of hydrogen and one mole of benzene from six moles of methane. Hydrogen can replace natural gas currently used for steam generation (resulting in zero or low carbon emissions), and benzene is condensed and sold as a readily-transported chemical commodity or fuel component. Pure methane is used as the example feed in this proposal. However, higher alkanes such as ethane and propane present in raw natural gas steams actually produce greater per-pass yields than methane, and their presence is desirable.

In one embodiment, raw field gas (such as but not limited to flare gas) can be used wherever it is available, as well as commercial-quality natural gas, making this process especially versatile.

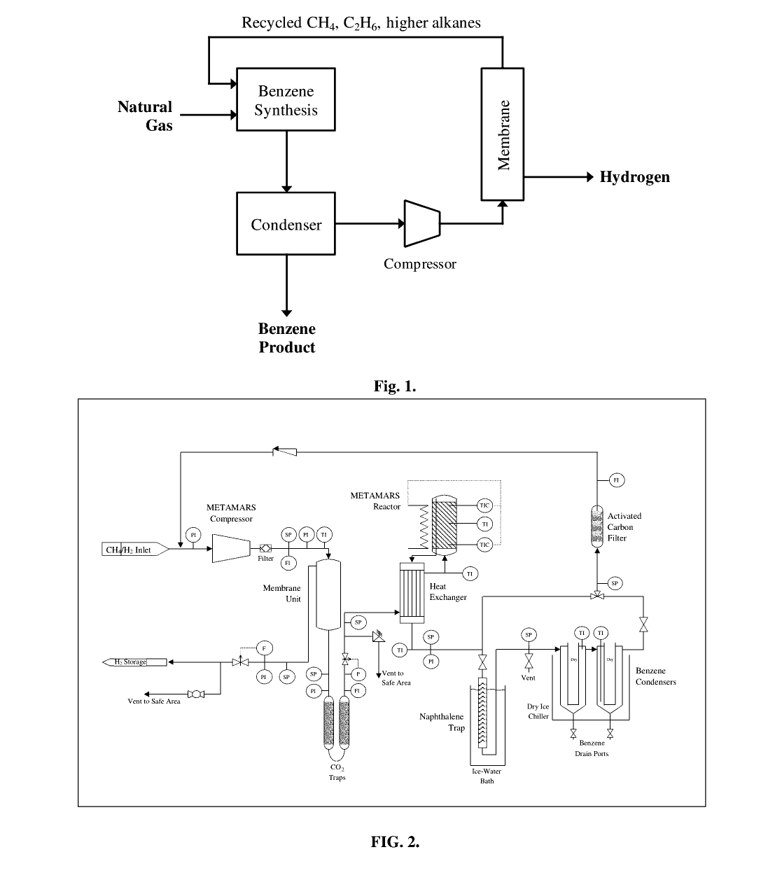

Reaction (1) does not proceed to completion in a single pass. Therefore, after condensing benzene, unreacted gases are separated from hydrogen and are recycled as shown in FIG. 1.

An example implementation of the process is illustrated below. Note that values may differ slightly due to use of pure methane rather than raw natural gas in the example case. The present case presumes production of 1600 GJ/hr of heat for steam generation according to the following reaction.

CH₄+2O₂→CO₂+2H₂O_{(g)} ΔH=−808 kJ@1500° C.(LHV) (2)

Reaction (2) requires consumption of about 1,065,000 standard cubic meters per day of methane to generate the required heat for steam generation. The equivalent natural gas heat load can be attained by combustion of hydrogen as shown in the following reaction.

H₂±0.5O₂→H₂O_{(g)} ΔH=−251 kJ@1500° C.(LHV) (3)

Reaction (3) requires the combustion of about 3,427,000 standard cubic meters per day of hydrogen, with zero carbon emissions. The production of hydrogen requires about 2,285,000 standard cubic meters of methane when prepared according to reaction (1).

Because reaction (1) above is endothermic, an additional heat input of about 427 GJ/hr is required for the aromatization reaction (conservatively assuming no available waste heat from other parts of the process). Therefore, an additional 610,000 standard cubic meters of methane per day is needed to produce hydrogen to supply heat input to the aromatization reaction (1), which equates to about 915,000 standard cubic meters per day of hydrogen. In total, about 2,900,000 standard cubic meters of methane would be required to support the process for replacement of natural gas at the current rate.

Based on the total hydrogen requirement and the corresponding natural gas input, the process would produce byproduct benzene according to reaction (1) in an amount of about 1,325 metric tons per day (or about 9,500 barrels per day). This represents about 1 percent of world demand and would generate gross revenues on the order of $800,000 per day based on January 2015 prices of about $600 per metric ton. Previous efforts to achieve alkane aromatization have resulted in significant reactor fouling and the need to frequently regenerate catalyst as a result of carbon deposition. Our work has showed that by controlling the amount of hydrogen present in the aromatization reactor, much longer operation could be achieved with only slight reduction in per-pass conversion.

Other embodiments include monetizing stranded natural gas (by converting alkanes to truck-transportable aromatics in remote locations, whereby hydrogen is used to provide process heat and power), or any other general hydrogen-from-natural gas applications (with co-production of benzene or aromatics to provide process heat and power plus byproduct sales). One of ordinary skill in the art will recognize numerous applications of the present invention from the disclosure of the central ideas presented herein.

Table 1 summarizes the current (base case) of direct methane combustion and the Natural Gas Decarbonization Process.

TABLE 1
Comparison of Current and Proposed
Methods for Steam Generation

| | Base Case | Proposed Case |
|---|---|---|
| | (Methane | (Hydrogen Combustion/ |
| Parameter | Combustion) | Benzene Production) |
| Methane Consumption | 1,065,000 | 2,285,000 |
| (m³/day) | | |
| Hydrogen Production | 0 | 4,342,000 |
| (m³/day) | | |
| Benzene Production | 0 | 1325 |
| (tonnes/day) | | |
| Daily Gross Benzene | 0 | $800,000 |
| Revenue | | |
| CO₂ Emissions | 2090 | 0 |
| (tonnes/day) | | |

In one embodiment, the process is a method of producing aromatic hydrocarbons from a natural gas feed, comprising the steps of passing the natural gas feed over a catalyst to form hydrogen gas and an aromatic hydrocarbon.

In one embodiment, the aromatic product is benzene. In one embodiment, the catalyst is a transition metal catalyst. In one embodiment, the catalyst is a transition metal catalyst doped on a zeolite catalyst.

In one embodiment the reaction is done between 250 and 950° C. In one embodiment, the hydrogen product is used as a fuel. In one embodiment, the hydrogen product is used to make steam and extract oil from oil sands. In one embodiment, the transition metal is molybdenum.

In other embodiments the metal is selected from iron, niobium, technetium, osmium, tantalum, rhenium, chromium, tungsten, vanadium, manganese and like transition group metals or mixtures thereof.

In one embodiment, the catalyst is a molybdenum on ZSM-5 catalyst. In one embodiment, the process is a low carbon dioxide emissions process for extracting hydrocarbons from oil sands, comprising passing a natural gas feed over a catalyst to form hydrogen gas and an aromatic hydrocarbon. In one embodiment, the hydrogen product is used to generate steam used to extract said hydrocarbons from oil sands.

The present invention relates to the upgrading of methane or natural gas into more valuable and readily transportable liquid hydrocarbons and hydrogen by-product. The invention specifically relates to a process for the conversion of hydrocarbon feedstock containing a major proportion of methane into liquids rich in aromatic hydrocarbons such as benzene, toluene, xylene, and naphthalene. One embodiment of the present invention comprises the non-oxidative aromatization of methane or natural gas using a metal-loaded crystalline aluminosilicate molecular sieve catalyst exhibiting a high selectivity to such aromatic hydrocarbons and the recycling of a portion of the hydrogen byproduct to extend the lifetime of the catalyst and hence the production of aromatic hydrocarbons prior to regeneration of the catalyst.

The market for benzene is large and diverse. While benzene demand for gasoline blending and as a solvent has declined, new uses as chemical intermediates have continued to increase. The current leading use of benzene (about one-half of all benzene usage) is in the manufacture of ethylbenzene, which is dehydrogenated to styrene used for production of plastics and synthetic rubbers. The styrene market exhibits a strong global growth rate of about 4.5% per year (ChemExpo Chemical Profile). Other significant uses of benzene are for manufacture of cumene in the production of phenolic resins and production of cyclohexane that is oxidized to adipic acid. Several other benzene-based processes for the manufacture of a variety of polyester resins, detergents from alkylbenzene, polymers, surface-active agents, and pesticides and herbicides represent the other main uses of benzene.

Benzene demand was 41 million metric tons in 2006. World production varies as a function of demand and variation in commodity prices related to benzene production from a suite of chemical byproducts. Although overall benzene production capacity slightly exceeds current demand, shifts in production sources are dictated by a range of other commodity prices. Therefore, certain benzene production processes are not economic when potential feed stocks are more-economically processed into other commodities. Much of the current benzene production comes from methods such as catalytic reforming of naphthenes followed by solvent extraction and fractional distillation, petroleum cracking, recovery from coke-oven gas, and recovery from benzene-toluene-xylene (BTX) manufacturing. The relative emphasis and deployment of each of these methods are shifted as market factors for each of the interrelated compounds change.

Benzene commodity pricing has roughly paralleled crude oil prices, rising to a high of $4.00/gallon late in 2006. Natural gas prices reached a high of about $10/thousand cubic feet (kcf) in early 2001 but have dropped to about $7-8/kcf at the well head currently. Prices of $2-3/kcf were typical prior to the significant run-up in 2000 (Energy Information Agency). Based on historical commodity pricing and linkage between natural gas and benzene pricing, the conversion of natural gas to benzene will result in a significant addition of value while also generating a very useful hydrogen byproduct.

The process for aromatic synthesis can generate about 5 gallons of benzene per 1,000 cubic feet of natural gas. The hydrogen byproduct will constitute a significant additional economic return. In cases where the natural gas would have been stranded, and thus commercially worthless unless converted to liquid, the advantage of utilizing the process would be even larger.

Beyond the recovery of “stranded” natural gas reserves, the successful terrestrial implementation of the benzene production process will result in a number of commercial advantages over existing benzene production routes. Direct benzene production from natural gas could supply a highly desirable, pure product without the current dependency on chemical or petroleum byproducts and the undesirable economic interaction with otherwise unrelated chemicals manufacturing. Greater economic stability and production management would be derived from a dedicated, high-selectivity benzene production process.

High-purity terrestrial benzene manufacturing would be facilitated by use of a natural gas feed following standard sulfur removal methods. Benzene product quality including refined, industrial, and ACS reagent grade products would be more easily produced from the relatively narrow range of natural gas purity compared to many other potential benzene feeds. In fact, impurities such as ethane generally present in natural gas will improve the yield of benzene from the process.

As illustrated above, recovery and delivery of benzene derived from natural gas in remote locations will result in significant generation of hydrogen. This byproduct hydrogen can be used to supply needed system energy, to provide power to the commercial electric grid through the use of gas turbines, internal combustion engines, fuel cells, or any other means, or to provide hydrogen for petrochemical processes. The clean-burning hydrogen combustion product (water) will have virtually no environmental impact on the region from which the natural gas is recovered. Thus, in addition to eliminating the discharge of flare combustion products from vast amounts of wasted natural gas, the recovery process itself is fueled by one of the cleanest possible energy sources. Furthermore, when combined with power generation equipment, the process of the invention provides a means to generate electric power with no greenhouse gas emissions to the atmosphere. Where specific situations permit, the excess hydrogen might also be used to upgrade heavier crude oil fractions to improve overall petroleum recovery and transportation characteristics.

With the implementation of the aromatic hydrocarbon process in industrialized areas, the available hydrogen can be used to satisfy the expected demand for fuel cells, hydrocracking to produce refined oils, and hydroprocessing to remove sulfur and nitrogen from petroleum.

It has been discovered by the present inventors that the process for aromatic hydrocarbons production is a useful and efficient for the production of high value liquid fuels and chemicals from methane or natural gas. By use of a recycling system, the majority of the methane and other natural gas components are converted into aromatic compounds, primarily benzene, over a molybdenum impregnated aluminosilicate molecular sieve catalyst, with typical temperatures of 600-800° C., pressures of 0.1 to 5 atmospheres, and Weight Hourly Space Velocity (WHSV) of 0.5-2.1 hr⁻¹. A fraction of the byproduct hydrogen is recycled after membrane separation into the aromatization reactor to inhibit the production of graphitic carbon which would otherwise rapidly deactivate the catalyst. Normally the hydrogen would be expected to suppress the aromatization reaction by the Principle of LeChatelier, but in practice benzene and other aromatic compound production continues at slightly reduced but still significant rates. In this counterintuitive manner, the lifetime of the catalyst is extended from about 10 hours to more than 100 hours before regeneration of the catalyst becomes necessary.

The resulting carbonaceous material which deactivates the catalyst contains some bonded hydrogen, making the carbonaceous material readily removable by pure hydrogen gas at a temperature of 600-800° C., a pressure of 0.1 to 5 atmospheres, and a WHSV of 0.1-0.2 hr⁻¹. The regeneration product is methane, which can be separated from the residual hydrogen using a membrane module and recycled into the feed. Full catalytic activity of the catalyst is restored using hydrogen regeneration.

In one embodiment the process is for the aromatization of alkane-containing gas, comprising: a reactor containing a metal-doped, crystalline aluminosilicate molecular sieve; activating the catalyst composition under a combined stream of the alkane-containing gas; contacting a feed stream comprising the gaseous hydrocarbon feed with the catalyst composition at hydrocarbon conversion conditions comprising a temperature greater than 600° C., a pressure greater than 0.01 bar; and producing aromatic compounds, primarily benzene, by majority conversion of the gaseous hydrocarbons by use of a recycling system.

In one embodiment the process is for the alkane-containing gas comprises at least approximately 85% methane.

In one embodiment the process is for the alkane-containing gas comprises a mixture of methane and nitrogen.

In one embodiment of the process the alkane-containing gas comprises methane, ethane, propane, and butane.

In one embodiment of the process the alkane-containing gas comprises nitrogen along with methane, ethane, propane, and butane.

In one embodiment of the process the alkane-containing gas comprises biogas.

In one embodiment of the process the feed comprises natural gas or any of the components thereof, such as methane, ethane, propane, or butane plus hydrogen.

In one embodiment of the process the feed comprises natural gas or any of the components thereof, such as methane, ethane, propane, or butane plus carbon monoxide.

In one embodiment of the process the feed comprises natural gas or any of the components thereof, such as methane, ethane, propane, or butane plus carbon dioxide.

In one embodiment of the process the feed comprises natural gas or any of the components thereof, such as methane, ethane, propane, or butane plus hydrogen plus carbon monoxide.

In one embodiment of the process the feed comprises natural gas or any of the components thereof, such as methane, ethane, propane, and butane plus hydrogen plus carbon dioxide.

In one embodiment of the process the catalyst composition is activated under a combined stream of natural gas or any of the components thereof, such as methane, ethane, propane, or butane plus hydrogen.

In one embodiment of the process the catalyst composition is regenerated with hydrogen.

In one embodiment of the process the catalyst composition is regenerated with oxygen.

In one embodiment of the process the catalyst composition is regenerated with air.

In one embodiment of the process the catalyst composition is regenerated with carbon dioxide.

In one embodiment of the process the catalyst composition is regenerated with hydrogen plus an inert component.

In one embodiment of the process the catalyst composition is regenerated with oxygen plus an inert component.

In one embodiment of the process the catalyst composition is regenerated with air plus an inert component.

In one embodiment of the process the catalyst composition is regenerated with carbon dioxide plus an inert component.

In one embodiment of the process the catalyst comprises a ZSM-5 molecular sieve.

In one embodiment of the process the catalyst comprises a ZSM-11 molecular sieve.

In one embodiment of the process the catalyst comprises a MCM-22 molecular sieve.

In one embodiment of the process the metal loaded on the catalyst comprises molybdenum.

In one embodiment of the process the metal loaded on the catalyst comprises ruthenium.

In one embodiment of the process the metal loaded on the catalyst comprises tungsten.

In one embodiment of the process the metal loaded on the catalyst comprises gallium.

In one embodiment of the process the metal loaded on the catalyst comprises rhenium.

In one embodiment of the process a secondary metal loaded on the catalyst comprises cobalt, indium, lanthanum, lithium, ruthenium, zinc, or zirconium.

In one embodiment of the process the aromatization reactor comprises of a set of parallel tubes to promote heat uptake by the reaction. In one embodiment of the process the aromatization reactor comprises multiple sets of parallel tubes to promote heat uptake by the reaction.

In other embodiments of the process the aromatization reactor comprises of an internal indirect heat exchange system to promote heat uptake by the reaction.

In one embodiment of the process the aromatization system is coupled to a carbon dioxide methanation system.

In one embodiment of the process system for the aromatization of alkane-containing gas, has a recycling reactor containing a metal-loaded, crystalline aluminosilicate molecular sieve; an activating the catalyst composition under a combined stream of methane and hydrogen or butane and hydrogen; where contacting a feed stream comprising at least 85% methane and at least 5% and no more than 15% hydrogen with the catalyst composition at hydrocarbon conversion conditions comprising a temperature from approximately 600° C. to approximately 900° C., a pressure of approximately 0.1 to approximately 5 atmospheres absolute, and a Weight Hourly Space Velocity (WHSV) of 0.5-3.0 h-1; produces aromatic compounds, primarily benzene, by majority conversion of the methane by use of a recycling system.

An embodiment of the process above wherein the catalyst comprises a ZSM-5 molecular sieve.

An embodiment of the process above wherein the catalyst comprises from about 2 to about 10 wt. % molybdenum.

An embodiment of the process above, wherein the catalyst comprises from about 0.5 to about 1 wt. % indium.

An embodiment of the process above, wherein the catalyst comprises a molecular sieve having a silica-to-alumina ratio of 30:1.

An embodiment of the process above, wherein the catalyst is activated under a combined stream which comprises at least 85% methane and at least 5% and no more than 15% hydrogen.

An embodiment of the process above, wherein the catalyst is activated under a combined stream which comprises at about 8.3% n-butane and about 92% hydrogen.

An embodiment of the process above, wherein the aromatization system is coupled to a carbon dioxide methanation system.

An embodiment of the process above, wherein the feed comprises natural gas with sulfur removed.

An embodiment of the process above, wherein the feed comprises ethane.

An embodiment of the process above, wherein the feed comprises propane.

An embodiment of the process above, wherein the feed comprises butane.

An embodiment of the process above, wherein the catalyst is regenerated with majority hydrogen at regeneration conditions comprising a temperature from approximately 600° C. to approximately 800° C., a pressure of approximately 1 to approximately 5 atmospheres absolute, and a Weight Hourly Space Velocity (WHSV) of 0.1-0.2 hr-1.

An embodiment of the process above wherein the aromatization system is coupled to a carbon dioxide methanation system.

An embodiment of the process above, wherein a substantial amount of the hydrogen product is used to generate electric power.

An embodiment of the process above, wherein a substantial amount of the hydrogen product is used for petrochemical processing.

An embodiment of the process above, wherein a substantial amount of the hydrogen product is used to generate electric power.

An embodiment of the process above, wherein a substantial amount of the hydrogen product is used for petrochemical processing.

In one embodiment, the Process for Aromatic Hydrocarbons Production from Methane invention comprises the contacting of the methane and hydrogen containing feed stream with a solid catalyst maintained at reaction conditions in a reaction zone. The feed stream may be methane or natural gas after removal of sulfur containing compounds, carbon dioxide, and carbon monoxide, and with the addition of hydrogen in one embodiment and with the nitrogen in other embodiments. One embodiment of the invention comprises the activation of the catalyst with 90% methane and 10% hydrogen at a temperature of 600-800° C., a pressure of 1-5 bar absolute, and a WHSV of 0.5-2.1 hr⁻¹. After separation using a membrane module, the aromatization process produces more than sufficient hydrogen to supply that added to the feed and that needed to regenerate the catalyst. Although the 600-800° C. temperature range is a preferred embodiment of the invention, higher temperatures increase the conversion of the endothermic methane to aromatics reaction.

In one embodiment, after passing through the benzene condensers, the residual gases consisting of unreacted feed, hydrogen byproduct, and small amounts of aromatic compounds are passed through a filter to trap any remaining aromatic compounds. After passing through the filter or trap, the unreacted feed and hydrogen are piped back to the compressor for combination with fresh feed and recycling through the membrane and aromatization reactor. Recycling results in complete conversion of methane, natural gas, or biogas into aromatic compounds.

In one embodiment, after passing through the benzene condensers, the residual gases consisting of unreacted feed, hydrogen byproduct, and small amounts of aromatic compounds are passed through an activated carbon filter to trap any remaining aromatic compounds. After passing through the activated carbon trap, the unreacted feed and hydrogen are piped back to the compressor for combination with fresh feed and recycling through the membrane and aromatization reactor. Recycling results in complete conversion of methane, natural gas, or biogas into aromatic compounds.

The invention encompasses all aspects of an end-to-end process for aromatic hydrocarbons production system. One embodiment of such a system would:

(1) aromatize the methane or natural gas to benzene, toluene, and naphthalene product,

(2) separate and recycle the unreacted methane or natural gas from most of the hydrogen produced, and

(3) use the hydrogen from the aromatization reaction for system energy, catalyst regeneration, or send it to a separate process for other useful purposes.

Referring now to FIG. 1, according to one embodiment of the invention, a system for converting methane or natural gas or biogas to aromatic liquid includes the non-oxidative conversion reactor connected to a heat exchanger. Methane, biogas, or natural gas and hydrogen along with unreacted feed and hydrogen byproduct are fed into a compressor which feeds a membrane separation unit. The membrane may be a hollow fiber membrane module such as the PA-1020-P1-2A-00 membrane from the Permea Division of Air Products, but can be any membrane system that separates carbon dioxide and the majority of hydrogen from methane or other gaseous hydrocarbons. In the membrane separation unit, excess hydrogen is removed by permeation from the feed, resulting in 5-15% hydrogen with the balance being methane or natural gas. The removed hydrogen is sent to storage or used in a separate process. The retentate from the membrane passes through an optional carbon dioxide trap if carbon dioxide has not already been removed from the feed. The treated feed passes through the heat exchanger where it is preheated by hot gases exiting the aromatization reactor.

In the aromatization reactor, the feed is typically heated to 600-800° C. (although good results may also be obtained at higher temperatures) and passed over a suitable aromatization catalyst producing benzene, toluene, naphthalene, and other aromatic compounds as well as hydrogen byproduct. The unreacted feed, aromatic compounds, and hydrogen exit the reactor through the heat exchanger, where substantial amounts of heat are recovered. The cooled gases then pass through an ice-water cooled naphthalene trap, where naphthalene and similar heavy aromatic compounds are converted into solid form to prevent clogging of the benzene and toluene condensers. The remaining gases pass into the benzene condensers which are cold fingers held at dry ice temperatures where the remaining aromatic compounds are solidified. The benzene condensers are held at dry ice temperatures because even solid benzene has a high vapor pressure at water ice temperatures. The benzene condensers are equipped with drains for product recovery once taken offline.

After passing through the benzene condensers, the residual gases consisting of unreacted feed, hydrogen byproduct, and small amounts of aromatic compounds are passed through an activated carbon filter to trap any remaining aromatic compounds. After passing through the activated carbon trap, the unreacted feed and hydrogen are piped back to the compressor for combination with fresh feed and recycling through the membrane and aromatization reactor. Recycling results in complete conversion of methane, natural gas, or biogas into aromatic compounds.

Solid naphthalene and similar products in the naphthalene trap can be recovered by melting and collection or by washing at room temperatures with liquid aromatics from the benzene condenser. Benzene, toluene, and other aromatics in the benzene condenser can be recovered by warming it to room temperatures and draining the liquid product into a suitable receiving container. Aromatic compounds in the activated carbon trap can be recovered by heating and passing the gases evolved through the benzene condenser at dry ice temperatures.

### Catalyst Preparation

A catalyst consisting of 6% Mo-0.7% In/HZSM-5 was prepared using the procedure outlined by Zhao (2002), modified to include incipient wetness impregnation of HZSM-5 which was previously converted to the ammonium form followed by drying at 80° C. and calcining at 700° C. A 11:1 hydrogen/n-butane treatment of 10% MoO₃ on HZSM-5 catalyst (which is also called H-MFI). A 10 g batch (“Small Batch”) and a 450 g batch (“Large Batch”) of the 6% Mo-0.7% In/HZSM-5 were prepared.

### Catalyst Testing

Small-scale testing of the methane-activated and n-butane-activated 6% Mo-0.7% In/HZSM-5 catalyst was accomplished using 78-80% methane, 9-10% carbon monoxide, and 10-12% hydrogen at 25 sccm (1500 scc/g-cat/hr=1.05 hr⁻¹ WHSV) at 700° C. The reactor consisted of a ½-inch o.d. stainless steel tube containing 1.0 g of catalyst supported on a stainless steel screen. The pressure was one bar gauge. Gas chromatography (GC) analysis of the inlet and exhaust gases was used to determine gas composition and monitor benzene production. A key discovery by the present inventors is that the presence of hydrogen in the feed methane greatly slows the deactivation of the catalyst while maintaining adequate rates of conversion of methane to aromatic compounds. FIG. 2 shows that the presence of hydrogen in the feed does not reduce the conversion to aromatic compounds as much as would be expected by the equation

6CH₄→C₆H₆+9H₂

which would predict that the conversion would be inversely proportional to the hydrogen concentration raised to the ninth power.

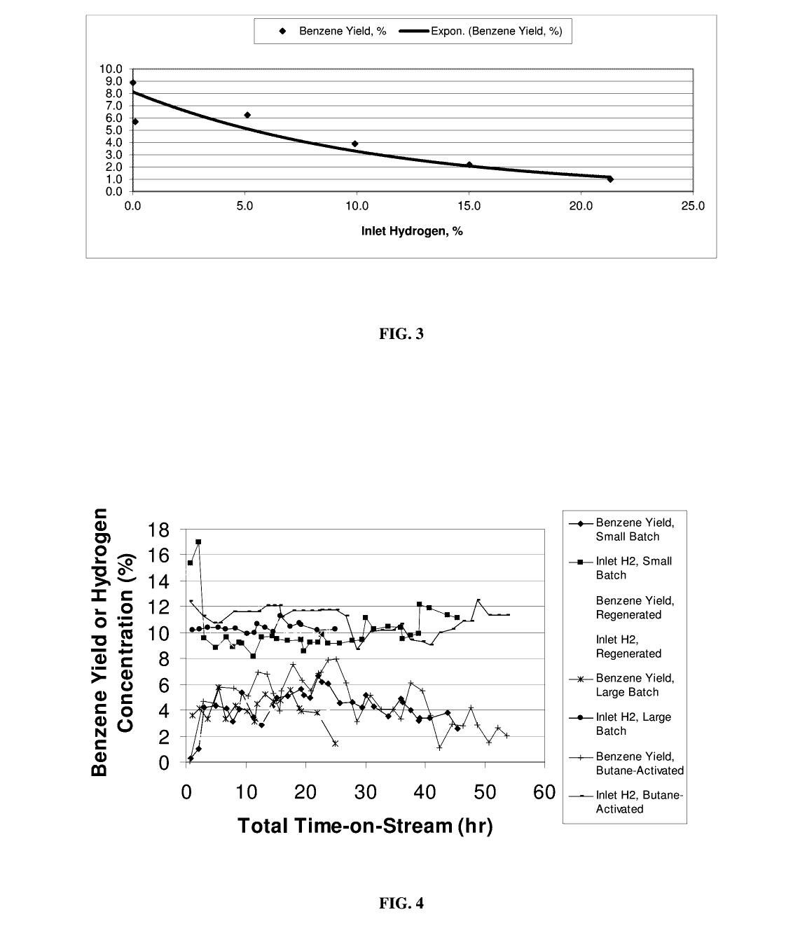

FIG. 3 summarizes the results of further catalyst tests that demonstrate this protective effect of hydrogen and compares the activity of the butane-activated catalyst to methane-activated catalyst, including hydrogen-regenerated Mo—In/HZSM-5. (To determine the approximate benzene yield, multiply the gaseous benzene concentration of the exhaust by six because it takes six methane molecules to make a benzene molecule.)

FIG. 3 shows that the butane-activated Mo—In/HZSM-5 has benzene yields similar to that of methane-activated catalyst and has a lifetime that is possibly longer than that of methane-activated catalyst under similar conditions. Further analysis of this data is presented in Table 2, which shows that the butane treatment is worth the additional effort because it results in a catalyst that is 41% more productive in making benzene during the first 25 hr TOS, is 90% as effective after 25 hr TOS, and has 25% less coke deposits, which is probably the source of the longer lifetime.

Table 2 also shows the results of hydrogen regeneration of the methane-activated and butane-activated catalysts, which was accomplished at 677° C., with a hydrogen flow rate of 28 sccm (=0.15 hr⁻¹ WHSV), and 1-2 bar gauge. Although the regeneration took longer for the butane-activated catalyst, the flow rate could be increased to shorten the regeneration time. The important observation is that the regeneration cycle was shorter than the catalyst lifetime. Thus, a system can be envisioned which has two aromatization reactors, one of which is producing

TABLE 2
Comparison of Methane-Activated Small Batch, Hydrogen-Regenerated Small Batch, Methane-
Activated Large Batch, and Butane-Activated Large Batch 6%Mo—0.7%In/HZSM-5.

| | | Average | | Average | | | | Coke | Grams |
|---|---|---|---|---|---|---|---|---|---|
| | | Benzene | | Benzene | | | | Removed, | of Coke |
| | Lifetime | Yield 0-25 | Avg. Inlet | Yield 25 | Avg. Inlet | H₂ Regen., | | % of Cat. | Deposited/ |
| Catalyst | (hr) | hr (%) | H₂ (%) | hr-on (%) | H₂ (%) | moles | Hours | Wt. | hr/g-cat. |
| Small Batch | 45.4 | 4.3 | 9.9 | 4.0 | 10.5 | 1.84 | 24.9 | 39.1 | 0.0088 |
| Regenerated | 23.7 | 3.4 | 10.2 | — | — | — | — | — | — |
| Small Batch | | | | | | | | | |
| Large Batch | 24.4 | 4.1 | 10.4 | — | — | — | — | — | — |
| Butane-Activated | 53.6 | 6.1 | 11.6 | 3.6 | 10.4 | 2.65 | 35.3 | 32.5 | 0.0061 |
| Large Batch | | | | | | | | | |

benzene, while the other is being regenerated. A separation membrane can readily accommodate the regeneration gases, which consist of hydrogen and a few percent of methane, by sending the hydrogen to storage or a separate process and the methane to the active aromatization reactor. The regenerated butane-activated catalyst was again tested for methane aromatization and showed good results, also shown in FIG. 2, with benzene yields similar to that found for the same catalyst over the first six hours.

These tests demonstrated the following characteristics of these catalysts:

A very long lifetime for methane aromatization, comparable to the longest available in the published literature. The addition of hydrogen to the methane feed gas inhibits the formation of graphitic coke that would otherwise eventually deactivate the catalyst and the n-butane treatment produces a very active form of the molybdenum carbide catalyst.

About 41% higher benzene yields (˜6.1% average) during the first 25 hr Time-on-Stream compared to methane-activated 6% Mo-0.7% In/HZSM-5, even in the presence of slightly higher hydrogen concentrations in the feed methane.

Easy regeneration of the catalyst over relatively short time periods with pure hydrogen. The coke formed in the presence of hydrogen in the feed contains hydrogen and is not refractory graphitic carbon that requires burning by oxygen for its removal.

Recovery of full catalyst activity after regeneration by hydrogen.

### EXAMPLES

A unit was designed to handle a total output of ˜130-300 grams of aromatic compounds per day. The METAMARS system converts CH₄ into C₆H₆, toluene, naphthalene, and H₂. The C₆H₆ is removed through a dry ice condenser system. Testing of the METAMARS reactor system showed the Mo—In/HZSM-5 catalyst to be rendered inactive by water vapor. Consequently a drying column was added to the methane feed. A carbon dioxide absorber was added to the inlet of the METAMARS reactor to prevent the formation of carbon monoxide by reaction with carbon on the catalyst. Carbon monoxide cannot easily be removed from the recycling system, and would build up system pressure. Further testing showed the METAMARS reactor must be designed to allow rapid heat transfer into the catalyst because the temperature is otherwise reduced to where the aromatics production rate is inadequate. A triple parallel reactor was designed and built from three one inch o.d. stainless steel tubes with plenums attached at the inlet and outlet to evenly distribute flow. The three reactors were loaded with 271 g of 6% Mo/HZSM-5. The indium promoter and n-butane activation were omitted to save preparation time. The reactors are heated with a ceramic heater and insulated to retain heat. A heat exchanger serves to minimize process heat loss.

Optimization tests show good conditions for methane aromatization at a feed of 10% H₂/90% CH₄ at a WHSV of 1.0 hr⁻¹, an aromatization reactor pressure of 1.2 bar gauge, 3 bar gauge membrane inlet pressure, and 700° C. aromatization reactor temperature. The liquid aromatics production rate of 4.1 g/hr rises to 4.5 g/hr when the proportionate fraction of the naphthalene and carbon traps cleanout (4.6 g) is added. Three duplicate runs were performed to verify the conclusion that 2.3 bar g membrane inlet pressure to be an excellent condition. Table 2 shows the results of the verification tests under these conditions. The first run was very successful and the system was very controllable. The liquid aromatics yield was 4.0 g/hr, similar to the first result for these conditions. Addition of the proportional amounts of aromatic products from the naphthalene condenser cleanout and the carbon trap boosts the yield to 5.4 g/hr or 130 g/day.

TABLE 3
Summary of Triple Parallel Reactor Brassboard Experiment Results

| | | Sample Time | METAMARS | | | GC | | | |
|---|---|---|---|---|---|---|---|---|---|
| Run | Wt. | (min after | Pressure | Flow Rate | Inlet | Benzene | Product | Total Run | Production |
| # | Catalyst (g) | run start) | (bar gauge) | (SLPM) | H₂ (%) | Yield (%) | Wt. (g) | Time (min) | Rate (g/hr) |
| 1 | 271 | 6 | 1.2 | 6.91 | 10.7 | | | | |
| | | 68 | 1.2 | 6.87 | 9.6 | | | | |
| | | 124 | 1.2 | 6.77 | 8.6 | | | | |
| | | 164 | 1.2 | 6.78 | 7.1 | | | | |
| | | 205 | 1.2 | 6.85 | 10.3 | 2.0 | | | |
| | | Averages | 1.2 | 6.8 | 9.3 | 2.0 | 15.0 | 227 | 4.0 |
| 2 | 271 | 50 | 1.2 | 6.75 | 7.0 | | | | |
| | | 76 | 1.2 | 6.78 | 8.0 | | | | |
| | | 108 | 1.2 | 6.81 | 6.1 | 1.9 | | | |
| | | Averages | 1.2 | 6.8 | 7.5 | 1.9 | 9.3 | 128 | 4.3 |
| Carbon Trap | | | | | | | 7.0 | | |
| 3 | 271 | 22 | 1.2 | 6.75 | 9.7 | | | | |
| | | 56 | 1.2 | 6.88 | 9.7 | | | | |
| | | 94 | 1.2 | 7.02 | 4.9 | | | | |
| | | 164 | 1.2 | 6.87 | 3.8 | | | | |
| | | 291 | 1.2 | 6.90 | 9.9 | | | | |
| | | 373 | 1.2 | 6.94 | 9.5 | | | | |
| | | 384 | 1.2 | 7.07 | 4.7 | | | | |
| | | 493 | 1.2 | 6.80 | 8.9 | 2.6 | | | |
| | | Averages | 1.2 | 6.9 | 7.6 | 2.6 | 23.6 | 510 | 2.8 |

| | |
|---|---|
| Cleanout (Naphthalene + Carbon Trap) | 4.1 |

The second verification run had a liquid aromatics yield of 4.3 g/hr, higher perhaps because the hydrogen concentration in the aromatics reactor inlet was a bit low at 7.5%. Addition of the naphthalene and carbon trap products in proportion to the liquid yield raises the aromatics production rate to 5.9 g/hr or 142 g/day.

A third run was performed in a full end-to-end operations for 8.5 hr. The liquid aromatics yield for this run was 2.8 g/hr. Addition of the naphthalene and carbon trap products raises it to 3.1 g/hr, somewhat less than the first two confirmatory runs. There was probably some unproductive time after two brief system upsets which caused the inlet hydrogen concentration of the aromatization reactor to rise to levels where no aromatics will be produced. Subtracting an hour of unproductive time gives a total production rate of 3.5 g/hr, still somewhat low. Nevertheless, Run 3 demonstrated that the METAMARS system can be run for long time periods and can be made to recover from system upsets and continue to produce products. With these and other tests, total run time of the Triple Tube Reactor with unregenerated 6% Mo/HZSM-5 was 103.4 hours, producing 364.3 grams of aromatic compounds.

### REFERENCES

The following patents and publications, and all patents and publications referred to above, are hereby incorporated by reference herein for all purposes.
1. U.S. Pat. No. 6,552,243 to Allison et al. (04/2003)
2. U.S. Pat. No. 7,019,184 to Allison et al. (03/2006)
3. Allison, J. D; S. Basso; M. Ledoux; P.-H. Cuong; and H. Wright, “Catalyst and Process for Aromatic Hydrocarbons Production from Methane,” International Patent Application WO 02/10099 A2, 2002.
4. Chen, L., J. Lin, H. C. Zeng, and K. L. Tan, “Non-oxidative methane conversion into aromatics on mechanically mixed Mo/HZSM-5 catalysts,” Catalyst Communications, 2, 201 (2001).
5. Ha, V. T. T.; L. V. Tiep; P. Meriaudeau; and C. Naccache, “Aromatization of methane over zeolite supported molybdenum: active sites and reaction mechanism,” J. Mol. Catal. A 181, 283 (2002).
6. Hamid, S. B. D.-A; J. R. Anderson; I. Schmidt; C. Bouchy; C. J. H. Jacobsen, and E. G. Derouane, “Effect of the activation procedure on the performance of Mo/H-MFI catalysts for the non-oxidative conversion of methane to aromatics,” Catal. Today 63, 461 (2000).
7. Lu, Yuan, Ding Ma, Zhusheng Xu, Zhijian Tian, Xinhe Bao, and Liwu Lin, “A high coking-resistance catalyst for methane aromatization”, Chem. Commun., (2001), 2048-2049.
8. Kim, Y. H., R. W. Bony, and E. Iglesia, “Genesis of methane activation sites in Mo-exchanged H-ZSM-5 catalysts,” Micr. Meso. Matl. 35-36, 495 (2000).
9. Kojima, R., S. Kikuchi, H. Ma, J. Bai, and M. Ichikawa, “Promotion effects of Pt and Rh on catalytic performances of Mo/HZSM-5 and Mo/HMCM-22 in selective methane-to-benzene reaction,” Catalysis Letters, 110 (2006), 15-20.
10. Ma, D.; Shu, Y.; Cheng, M.; Xu, Y.; and Bao, X., “On the Induction Period of Methane Aromatization over Mo-Based Catalysts,” J. Catalysis, 194, 105 (2000).
11. Ma, D.; Shu, Y.; Han, X.; Liu, X.; Xu, Y.; and Bao, X., “Mo/HMCM-22 Catalysts for Methane Dehydroaromatization: A Multinuclear MAS NMR Study,” J. Phys. Chem. B, 105, 1786 (2001).
12. Ma, D.; Han, X.; Xie, S.; Bao, X.; Hu, H.; and Au-Yeung, S. C. F., “An Investigation of the Roles of Surface Aluminum and Acid Sites in the Zeolite MCM-22,” Chem. Eur. J., 8, 162 (2002).
13. Ohnishi, R.; S. Liu, Q. Dong, L. Wang, and M. Ichikawa, “Catalytic dehydrocondensation of methane with CO and CO₂ toward benzene and naphthalene on Mo/HZSM-5 and Fe/Co-modified Mo/HZSM-5,” J. Catal., 182, 92 (1999).
14. Shu, Y. and Ichikawa, M., “Catalytic Dehydrocondensation of methane towards benzene and naphthalene on transition metal supported zeolite catalysts: templating role of zeolite micropores and characterization of active metallic sites,” Catal. Today, 71, 55 (2001).
15. Shu, Y.; Ohnishi, R.; and Ichikawa, M., “Pressurized Dehydrocondensation of Methane toward Benzene and Naphthalene on Mo/HZSM-5 Catalyst: Optimization of Reaction Parameters and Promotion by CO₂ Addition,” J. Catal., 206, 134 (2002).
16. Shu, Y.; Ohnishi, R.; and Ichikawa, M., “A Highly Selective and Coking-Resistant Catalyst for Methane Dehydrocondensation,” Chem. Lett., 2002, 418 (2002b).
17. Wang, L., Y. Xu, S.-T. Wong, W. Cui, and X. Guo, “Activity and stability enhancement of Mo/HZSM-5-based catalysts for methane non-oxidative transformation to aromatics and C₂ hydrocarbons: Effect of additives and pretreatment conditions,” Appl. Catal. A, 152, 173 (1997).
18. Zhang, Y.-P.; Wang, D.-J.; Fei, J.-H.; and Zheng, X.-M., “Methane aromatization under O₂-free conditions on zinc modified Mo/HZSM-5 catalyst,” React. Kin. Catal. Lett., 74, 151 (2001).
19. Zhou, X.; Xu, Z; Zhang, T.; and Lin, L., “The chemical status of indium in indium impregnated HZSM-5 catalysts for the SCR of NO with CH4,” J. Mol. Cat. A, 122, 125 (1997).
20. Zhou, X.; Zhang, T.; Xu, Z; and Lin, L., “Selective catalytic reduction of nitrogen monoxide with methane over impregnated In/HZSM-5 in the presence of excess oxygen,” Catal. Lett., 40, 35 (1996).
21. Zhao, J., X. Wang, T. Zhang, L. Li, N. Li, M. Zheng, and L. Lin, “Dehydro-Oligomerization of Methane to Benzene and Naphthalene without Adding Oxygen: Promotional Effect of In over Mo/HZSM-5 Catalyst,” ACS Fuel Chemistry Division Preprints, 47, 90 (2002).

What is claimed is:

1. A process for the aromatization of alkane-containing gas, comprising:
reacting a mixture of an alkane gas and hydrogen over a metal-loaded, crystalline aluminosilicate molecular sieve catalyst; at temperatures between 600° C. to approximately 900° C., a pressure of approximately 0.1 to approximately 5 atmospheres absolute, and a Weight Hourly Space Velocity (WHSV) of 0.5-3.0 h-1 to form aromatic compounds which are condensed and hydrogen.

2. The process of claim 1 where the alkane-containing gas comprises at least approximately 85% methane.

3. The process of claim 1 where the alkane-containing gas comprises methane, ethane, propane, and butane.

4. The process of claim 1 where the alkane-containing gas comprises biogas.

5. The process of claim 1 where the feed comprises natural gas or any of the components thereof, such as methane, ethane, propane, or butane plus hydrogen.

6. The process of claim 1 where the feed comprises natural gas or any of the components thereof, such as methane, ethane, propane, and butane combined with hydrogen and one or more of carbon dioxide, carbon monoxide and nitrogen.

7. The process of claim 1 where the catalyst composition is regenerated with hydrogen.

8. The process of claim 1 where the catalyst composition is regenerated with a gas selected from the group consisting of oxygen, air, carbon dioxide or hydrogen.

9. The process of claim 1 where the catalyst composition is regenerated with a gas selected from the group consisting of oxygen, air, carbon dioxide or hydrogen combined with an inert gas.

10. The process of claim 1 where the molecular sieve for the catalyst is selected from the group consisting of a metal doped ZSM-5, ZSM-11 or MCM-22 molecular sieve catalyst.

11. The process of claim 1 where the metal loaded on the molecular sieve is selected from the group consisting of molybdenum, ruthenium, tungsten, gallium or rhenium,

12. The process of claim 1 where a secondary metal loaded on the catalyst comprises cobalt, indium, lanthanum, lithium, ruthenium, zinc, or zirconium.

13. The process of claim 1 where after forming aromatic compounds which are condensed, the residual gases consisting of unreacted feed, hydrogen byproduct, and small amounts of aromatic compounds are passed through a filter to trap any remaining aromatic compounds. After passing through the filter or trap, the unreacted feed and hydrogen are piped back to the compressor for combination with fresh feed and recycling through the membrane and aromatization reactor.

14. The process of claim 1 where the aromatization system is coupled to a carbon dioxide methanation system.

15. The process of claim 1 where the catalyst is activated with a combined stream of methane and hydrogen or butane and hydrogen; where contacting a feed stream comprising at least 85% methane and at least 5% and no more than 15% hydrogen

16. The process of claim 1 where the catalyst comprises from about 2 to about 10 wt. % molybdenum.

17. The process of claim 1 where the catalyst comprises from about 0.5 to about 1 wt. % indium.

18. The process of claim 1 where the catalyst comprises a molecular sieve having a silica-to-alumina ratio of 30:1.

19. The process of claim 1 where the catalyst is activated under a combined stream which comprises at least 85% methane and at least 5% and no more than 15% hydrogen.

20. The process of claim 1 where the catalyst is activated under a combined stream which comprises at about 8.3% n-butane and about 92% hydrogen.

21. The process of claim 1 where the catalyst is regenerated with majority hydrogen at regeneration conditions comprising a temperature from approximately 600° C. to approximately 800° C., a pressure of approximately 1 to approximately 5 atmospheres absolute, and a Weight Hourly Space Velocity (WHSV) of 0.1-0.2 hr-1.

22. The process of claim 1 where a substantial amount of the hydrogen product is used to generate electric power.

23. The process of claim 1 where a substantial amount of the hydrogen product is used for petrochemical processing, feed fuel cells or chemical processes to make chemicals and fuels.

24. A low carbon dioxide emissions method for extracting hydrocarbons from oil sands, comprising:
passing a natural gas feed over a catalyst to form hydrogen gas and an aromatic hydrocarbon; and
using the hydrogen gas to generate steam used to extract said hydrocarbons from oil sands.

25. The method of claim 24, wherein the aromatic hydrocarbon is benzene.

26. The method of claim 24, wherein the catalyst is a transition metal catalyst.

27. The method of claim 24, wherein the catalyst is a transition metal catalyst doped on a zeolite catalyst.

28. The method of claim 24, wherein the reaction is done between 250 and 950° C.

29. The method of claim 24, wherein the hydrogen gas is used as a fuel.

30. The method of claim 24, where the hydrogen as is used to make steam and extract oil from oil sands.

31. The method of claim 16, wherein the transition metal is molybdenum.

32. The method of claim 27, where the catalyst is a molybdenum on ZSM-5 catalyst.

33. The process of claim 24 where
the aromatic compound is condensed,
the residual gases consisting of unreacted feed, hydrogen byproduct, and small amounts of aromatic compounds are passed through a filter to trap any remaining aromatic compounds, and,
after passing through the filter or trap, the unreacted feed and hydrogen are passed back to the compressor for combination with fresh feed and recycling.
